(19) United States
(12) Patent Application Publication
(10) Pub. No.: US 2017/0298026 A1
(43) Pub. Date: Oct. 19, 2017

(54) ALL PURPOSE RAFT AGENT

(71) Applicant: Commonwealth Scientific and Industrial Research Organisation, Acton, Australian Capital Territory (AU)

(72) Inventors: John Tsanaktsidis, Lysterfield (AU); James Gardiner, Wantirna (AU)

(21) Appl. No.: 15/516,622

(22) PCT Filed: Oct. 5, 2015
(86) PCT No.: PCT/AU2015/050602

§ 371 Date: Apr. 3, 2017

(30) Foreign Application Priority Data
Oct. 9, 2014 (AU) 2014904038

(51) Int. Cl. C07D 231/12; C08F 2/38

(52) U.S. Cl. CPC C07D 231/12; C08F 2/38

(57) ABSTRACT

The present invention relates to a RAFT agent of formula (I) where R₁, R₂ and R₃ are each independently selected from H and optionally substituted alkyl.

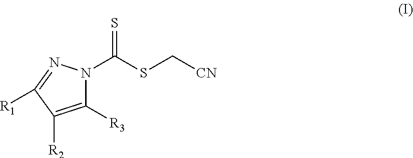

### FIELD OF THE INVENTION

The present invention relates generally to RAFT polymerisation. More specifically, the invention relates to a particular class of RAFT agent, to polymers prepared using the RAFT agent and to a method of preparing polymer using the RAFT agent.

### BACKGROUND OF THE INVENTION

Reversible addition-fragmentation chain transfer (RAFT) polymerisation, as described in International Patent Publication No. WO 98/01478, is a polymerisation technique that exhibits the characteristics associated with living polymerisation. Living polymerisation is generally considered in the art to be a form of chain polymerisation in which irreversible chain termination is substantially absent. An important feature of living polymerisation is that polymer chains will continue to grow while monomer and the reaction conditions to support polymerisation are provided. Polymers prepared by RAFT polymerisation can advantageously exhibit a well defined molecular architecture, a predetermined molecular weight and a narrow molecular weight distribution or low dispersity ( [symbol] ).

RAFT polymerisation is believed to proceed under the control of a RAFT agent according to a mechanism which is simplistically illustrated below in Scheme 1.

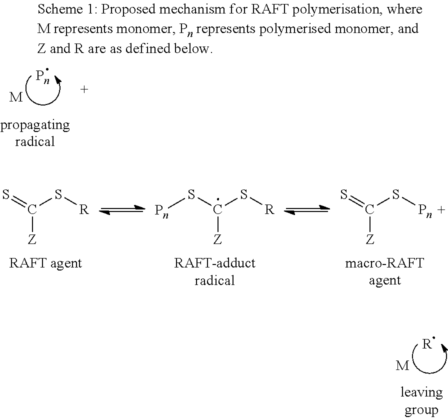

With reference to Scheme 1, R represents a group that functions as a free radical leaving group under the polymerisation conditions employed and yet, as a free radical leaving group, retains the ability to reinitiate polymerisation. Z represents a group that functions to convey suitable reactivity to the C═S moiety in the RAFT agent towards free radical addition without slowing the rate of fragmentation of the RAFT-adduct radical to the extent that polymerisation is unduly retarded. The ability for both R and Z to function in this way for a given agent is known to be influenced by the nature of the monomer to be polymerised and the polymerisation conditions.

In practice, the R and Z groups of a RAFT agent for use in a given polymerisation reaction are typically selected having regard to the type of monomers that are to be polymerised. For example, it is known in the art that Z groups that afford dithiocarbamate and xanthate RAFT agents can in general be used for controlling the polymerisation of monomers that produce relatively unstabilised propagating radicals (i.e. less activated monomers (LAM's) such as vinyl esters and vinyl amides), whereas Z groups that form dithioester and trithiocarbonate RAFT agents can in general be used for controlling the polymerisation of monomers that produce relatively stabilised propagating radicals (i.e. more activated monomers (MAM's) such as styrenes, acrylates, acrylamides, methacrylates and methacrylamides). In other words, most RAFT agents will generally be unsuitable for use in controlling the polymerisation of both less activated and more activated monomers (i.e. monomers having markedly disparate reactivities e.g. styrene and vinyl acetate).

Having said this, under limited circumstances a RAFT agent may be used to polymerise a mixture of less activated and more activated monomers. In that case, those skilled in the art will appreciate that provided the reactivity and mole ratios of the selected monomers are suitable, a RAFT agent may be used to prepare a statistical (or random) copolymer comprising the polymerised residues of less activated and more activated monomers. Those skilled in the art will also appreciate that one practical upshot of this is that to date it has proven difficult to prepare block copolymers by RAFT polymerisation that are derived from less activated and more activated monomers (e.g. a vinyl acetate-acrylate block copolymer).

WO 2010/083569 discloses RAFT agents having the potential to polymerise monomers with disparate reactivities. The disclosed RAFT agents require the presence of a Lewis base moiety that upon interaction with a Lewis acid moiety form a RAFT agent Lewis adduct. The process of association or disassociation of the Lewis acid moiety with the Lewis base moiety has the effect of “switching” the reactivity of the RAFT agent such that in its Lewis adduct form it can function to polymerise more activated monomers, whereas in its “free base” form it can function to polymerise less activated monomers. While effectively providing a RAFT agent capable of polymerising both less and more activated monomers, to function in this way use of the RAFT agents require the additional process step of being “switched” either into an adduct or free base form.

Although there are numerous advantages to be gained by using conventional RAFT agents to form polymer, it would nonetheless be desirable to provide a RAFT agent that offered further or new utility relative to those currently known.

### SUMMARY OF THE INVENTION

The present invention provides a RAFT agent of formula (I)

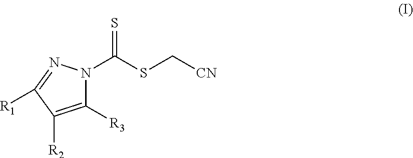

where R₁, R₂ and R₃ are each independently selected from H and optionally substituted alkyl (for example optionally substituted C₁-C₁₂ alkyl).

It has now been surprisingly found that a RAFT agent of formula (I) can advantageously be used to polymerise both LAM's and MAM's. Notably, such RAFT agents can function to polymerise both LAM's and MAM's without the need to be structurally modified or “switched” in any way. The RAFT agents can advantageously be used to polymerise both LAM's and MAM's to afford polymers, including block copolymers, having a low dispersity ( [symbol] ). For example, polymer formed using a RAFT agent of formula (I) can be provided with a dispersity ( [symbol] ) of less than 1.5, or less than 1.4, or less than 1.3, or less than 1.2 or less than 1.1.

Without wishing to be limited by theory, it is believed the specific structural features of a RAFT agent of formula (I) provides for a unique reactivity profile that enables the agent to polymerise monomers having disparate reactivities. RAFT agents of formula (I) are therefore particularly versatile and not only simplify the range of RAFT agents a person skilled in the art would typically have on hand to polymerise monomers of disparate reactivities, but they provide an opportunity to conveniently prepare block copolymers by RAFT polymerisation comprising polymer blocks derived from monomers of disparate reactivities.

The present invention therefore also provides polymer of formula (II) or (III)

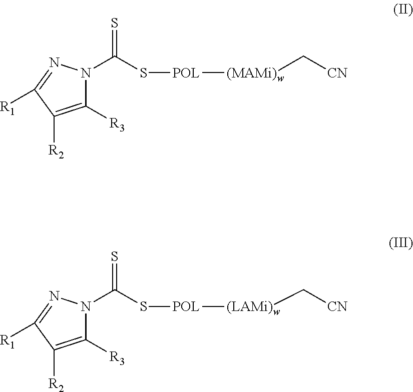

where R₁, R₂ and R₃ are each independently selected from H and optionally substituted alkyl (for example optionally substituted C₁-C₁₂ alkyl), POL in formula (II) is a polymer chain comprising (PMAM)ₓ-(PLAM)_{y}*, POL in formula (III) is a polymer chain comprising (PLAM)_{y}-(PMAM)ₓ*, MAMi and PMAM are each independently made up of one or more RAFT reaction monomer residue units derived from one or more ethylenically unsaturated monomers of formula (IV), LAMi and PLAM are each independently made up of one or more RAFT reaction monomer residue units derived from one or more ethylenically unsaturated monomers selected from formula (V), w, x and y are each independently 0 or 1 provided at least one of x or y is 1, and * denotes the point of covalent coupling to (i) MAMi or LAMi when present, or (ii) —CH₂—CN when MAMi or LAMi are not present,

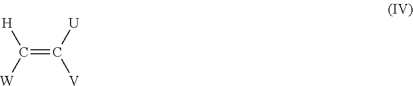

where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain,

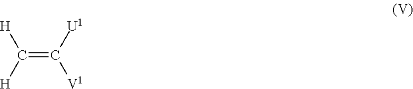

where U¹ is selected from H, C₁-C₄ alkyl or halogen; V¹ is halogen or of the form O-G where G is selected from —C(O)R¹ and —R¹, or V¹ is of the form NGG^{a} where G is as defined above and G^{a} is selected from H and R¹, G and G^{a} form together with N a heterocyclic ring, or V¹ is of the form CH₂G^{b} where G^{b} is selected from H, R¹, OH, OR¹, NR¹₂, PR¹₂, P(O)R¹₂, P(OR¹)₂, SR¹, SOR¹, and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

In the context of formula (II) and (III), by w, x and y being independently 0 or 1 is meant the associated moiety is either present or not present, with the presence or absence of a given moiety not effecting the presence or absence of another moiety.

In one embodiment, w in formula (II) is 1. In that case, MAMi is present and may represent from 1 to about 20, or from 1 to about 15, or from 1 to about 10 RAFT reaction monomer residue units derived from one or more ethylenically unsaturated monomers of formula (IV).

In another embodiment, w in formula (III) is 1. In that case, LAMi is present and may represent from 1 to about 20, or from 1 to about 15, or from 1 to about 10 RAFT reaction monomer residue units derived from one or more ethylenically unsaturated monomers of formula (IV).

If present, MAMi and LAMi may therefore be seen to represent a relatively low number of RAFT reaction monomer residue units.

In formula (II) and (III), PMAM and PLAM will generally each independently represent at least about 10, or at least about 15, or at least about 20 RAFT reaction monomer residue units of one or more ethylenically unsaturated monomers of formula (IV) and (V), respectively.

The PMAM and PLAM in formula (II) and (III) may therefore also each be described as a polymer chain or block made up of RAFT polymerised monomer residue units derived from one or more ethylenically unsaturated monomers of formula (IV) and (V), respectively.

In formula (II) and (III), at least PMAM or PLAM must be present (i.e. x or y must be 1).

Where x and y in formula (II) or (III) are both 1, it will be appreciated that POL represents a polymer chain comprising a block copolymer, that block copolymer comprising (i) a polymer block made up of RAFT reaction monomer residue units derived from one or more ethylenically unsaturated monomers of formula (IV), and (ii) a polymer block made up of RAFT reaction monomer residue units derived from one or more ethylenically unsaturated monomers of formula (V).

Where x and y in formula (II) or (III) are both 1, POL may therefore represent a polymer chain comprising a block copolymer, the block copolymer comprising (LAM)-(MAM) in the case of formula (II), or (MAM)-(LAM) in the case of formula (III).

In one embodiment, x and y in formula (II) and/or (III) are both 1.

The present invention also provides a method of preparing polymer, the method comprising polymerising under the control of a RAFT agent of formula (I) one or more ethylenically unsaturated monomers of formula (IV) and/or (V)

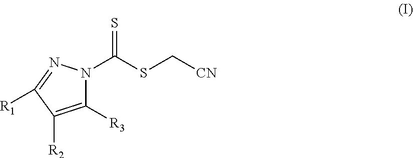

where R₁, R₂ and R₃ are each independently selected from H and optionally substituted alkyl (for example optionally substituted C₁-C₁₂ alkyl),

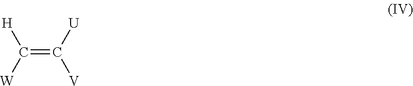

where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain,

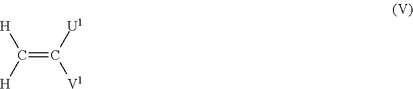

where U¹ is selected from H, C₁-C₄ alkyl or halogen; V¹ is halogen or of the form O-G where G is selected from —C(O)R¹ and —R¹, or V¹ is of the form NGG^{a} where G is as defined above and G^{a} is selected from H and R¹, G and G^{a} form together with N a heterocyclic ring, or V¹ is of the form CH₂G^{b} where G^{b} is selected from H, R¹, OH, OR¹, NR¹₂, PR¹₂, P(O)R¹₂, P(OR¹)₂, SR¹, SOR¹, and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

According to this method of preparing polymer, in one embodiment the method may be preceded by reacting under the control of the RAFT agent of formula (I) one or more ethylenically unsaturated monomers of formula (IV) or (V) to form a macro-RAFT agent, wherein one or more ethylenically unsaturated monomers of formula (IV) or (V) are polymerised under the control of the so formed macro-RAFT agent to form the intended polymer, and wherein monomer reacted in the preceding step is not of the same formula of monomer polymerised in the step of preparing the intended polymer.

The present invention therefore also provides a method of preparing polymer, the method comprising:

(i) reacting under the control of a RAFT agent of formula (I) one or more ethylenically unsaturated monomers of formula (IV) or (V) to form a macro-RAFT agent; and

(ii) polymerising under the control of the macro-RAFT agent formed in step (i) one or more ethylenically unsaturated monomers of formula (IV) or (V) so as to form the polymer,

wherein monomer polymerised in step (ii) is not of the same formula of monomer reacted in step (i).

The present invention also provides a method of preparing polymer, the method comprising: reacting under the control of a RAFT agent of formula (I) a mixture of one or more ethylenically unsaturated monomers of formula (IV) and one or more ethylenically unsaturated monomers of formula (V), wherein the monomer mixture comprises a higher concentration of monomer of formula (IV) or (V).

In one embodiment, the monomer mixture comprises as the higher monomer concentration monomer of formula (IV) or (V) being at least 60 mol %, or at least 70 mole %, or at least 80 mol %, or at least 90 mol %, or at least 95 mol %, relative to the lower monomer concentration monomer of formula (IV) or (V).

In another embodiment, monomer of formula (IV) presents as the higher concentration monomer.

In a further embodiment, monomer of formula (V) presents as the higher concentration monomer.

The present invention further provides a method of preparing block copolymer that comprises a polymer block derived from one or more ethylenically unsaturated monomers of formula (IV) and a polymer block derived from one or more ethylenically unsaturated monomers of formula (V), the method comprising:

(i) polymerising under the control of a RAFT agent of formula (I) one or more ethylenically unsaturated monomers of formula (IV) or (V) to form a macro-RAFT agent; and

(ii) polymerising under the control of the macro-RAFT agent formed in step (i) one or more ethylenically unsaturated monomers of formula (IV) or (V) so as to form the block copolymer,

wherein monomer polymerised in step (ii) is not of the same formula of monomer polymerised in step (i),

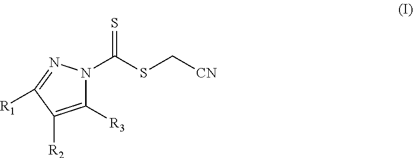

where R₁, R₂ and R₃ are each independently selected from H and optionally substituted alkyl (for example optionally substituted C₁-C₁₂ alkyl),

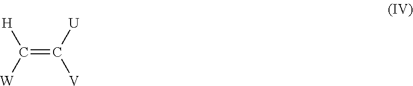

where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain,

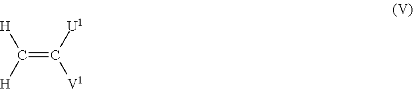

where U¹ is selected from H, C₁-C₄ alkyl or halogen; V¹ is halogen or of the form O-G where G is selected from —C(O)R¹ and —R¹, or V¹ is of the form NGG^{a} where G is as defined above and G^{a} is selected from H and R¹, G and G^{a} form together with N a heterocyclic ring, or V¹ is of the form CH₂G^{b} where G^{b} is selected from H, R¹, OH, OR¹, NR¹₂, PR¹₂, P(O)R¹₂, P(OR¹)₂, SR¹, SOR¹, and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

According to the method of preparing block copolymer, in one embodiment step (i) may be preceded by reacting under the control of the RAFT agent of formula (I) one or more ethylenically unsaturated monomers of formula (IV) or (V) to form a macro-RAFT agent which is then used as the RAFT agent in step (i), wherein monomer reacted in this step preceding step (i) is not of the same formula of monomer polymerised in step (i).

The present invention therefore also provides a method of preparing block copolymer that comprises a polymer block derived from one or more ethylenically unsaturated monomers of formula (IV) and a polymer block derived from one or more ethylenically unsaturated monomers of formula (V), the method comprising:

(i) reacting under the control of a RAFT agent of formula (I) one or more ethylenically unsaturated monomers of formula (IV) or (V) to form a macro-RAFT agent;

(ii) polymerising under the control of the macro-RAFT agent formed in step (i) one or more ethylenically unsaturated monomers of formula (IV) or (V) to form a macro-RAFT agent; and

(iii) polymerising under the control of the macro-RAFT agent formed in step (ii) one or more ethylenically unsaturated monomers of formula (IV) or (V) to form the block copolymer,

wherein monomer polymerised in step (iii) is not of the same formula of monomer polymerised in step (ii), and monomer polymerised in step (ii) is not of the same formula of monomer reacted in step (i).

RAFT agents in accordance with the invention have been found to be particularly effective at polymerising monomer that provides for secondary incipient radicals. In one embodiment, ethylenically unsaturated monomer referred to or used in accordance with the invention has a structure that provides for a secondary incipient radical.

Accordingly, in one embodiment ethylenically unsaturated monomers of formula (IV) and (V) are selected such that they provide for a secondary incipient radical. For example, ethylenically unsaturated monomers of formula (IV) may be selected from styrenes, acrylates and acrylamides, and ethylenically unsaturated monomers of formula (V) may be selected from vinyl esters and vinyl amides.

These and other aspects of the invention are described in more detail below.

### DETAILED DESCRIPTION OF THE INVENTION

The present invention provides a RAFT agent of formula (I)

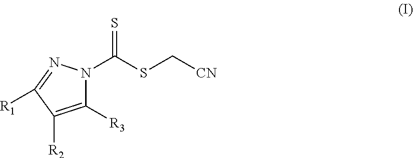

where R₁, R₂ and R₃ are each independently selected from H and optionally substituted alkyl.

In one embodiment, R₁, R₂ and R₃ are each independently selected from H and optionally substituted C₁-C₁₂ alkyl.

In a further embodiment, R₁, R₂ and R₃ are each independently selected from H and CH₃.

In another embodiment, R₁ and R₃ are each CH₃, and R₂ is H.

Those skilled in the art will appreciate the structure of formula (I) may be described as being a RAFT agent. By being a “RAFT agent” is meant the agent is capable of participating in RAFT polymerisation reaction. A RAFT polymerisation reaction is believed to proceed under the control of a RAFT agent according to the mechanism outlined in Scheme 1 (above).

Reference herein to a “RAFT agent” is intended to embrace a macro-RAFT agent.

In a RAFT polymerisation reaction, one or more ethylenically unsaturated monomers are believed to react under the control of the RAFT agent. By reacting or being polymerised “under the control” of the RAFT agent is meant that reaction of monomer proceeds via a reversible addition-fragmentation chain transfer mechanism.

Those skilled in the art will appreciate that RAFT agents can advantageously provide excellent control over the reaction process between the agent and monomer. So much so that reaction between a RAFT agent and monomer can provide for a relatively accurate and predetermined number of monomer residue units that become inserted into the RAFT agent.

Inserted monomer residue units referred to herein are therefore those derived from monomer that participates in a RAFT reaction to become covalently bound to the RAFT agent.

For example, monomer may undergo reaction with a RAFT agent (or fragment thereof) whereby a relatively low number of monomer residue units (e.g. 5) are inserted, or a relatively high number of monomer residue units (e.g. 500 or 1000) are inserted.

Where a given RAFT agent (or fragment thereof) undergoes reaction with monomer and a low or high number of monomer residue units are inserted, the reaction between the RAFT agent and monomer is commonly referred to as a RAFT “polymerisation” reaction or process.

Accordingly, reference herein to polymerising or reacting monomer under the control of a RAFT agent (including a macro-RAFT agent) is intended to mean that monomer undergoes reaction with the RAFT agent such that one or more monomer residue inserts into the RAFT agent via a reversible addition-fragmentation chain transfer mechanism.

Where at least 10, or at least 15, or at least 20 RAFT reaction monomer residue units are inserted it may be convenient to refer to the collective of monomer residue units as a polymer chain. In that case, it can be convenient refer to polymerising monomer, or monomer being polymerised, under the control of the RAFT agent.

Where less than 20, or less than 15, or less than 10 RAFT reaction monomer residue units are inserted to the RAFT agent it may be convenient to collectively or individually refer to the monomer residue unit(s) as an oligomer or simply as monomer residue unit(s). In that case, it can be convenient refer to reacting monomer, or monomer being reacted, under the control of the RAFT agent.

As will be discussed in more detail below, it has now been surprisingly found that a RAFT agent of formula (I) can advantageously be used to polymerise a diverse range of monomers, including those commonly referred to as LAM's and MAM's. In other words, RAFT agents in accordance with the invention may be described as a true general all-purpose RAFT agent.

Synthetic techniques known by those skilled in the arts can advantageously be applied to prepare RAFT agents of formula (I). For example, a general synthetic approach may comprise nitrogen nucleophilic addition to carbon disulphide followed by subsequent alkylation of the resulting dithiocarbazate with a cyanomethyl halide. A general reaction mechanism according to this approach is outlined below in Scheme 2.

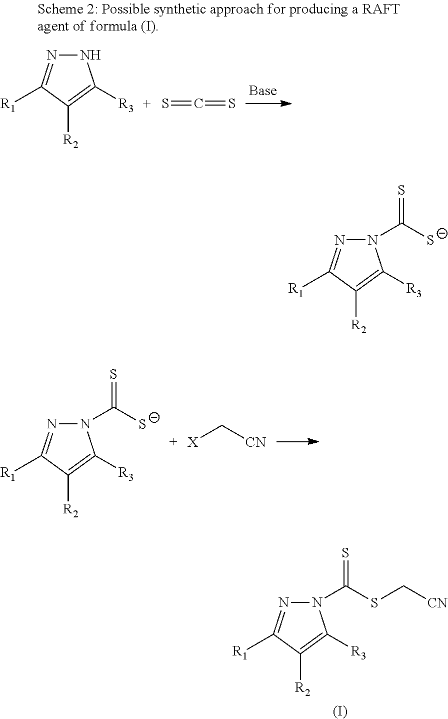

Synthetic methodology similar to that outlined in Scheme 2 above may be found in published scientific journals, for example Castro et al, J. Org. Chem., 1984, 49, 863, the contents of which are incorporated herein by reference.

The procedure for the first step, synthesis of potassium 3,5-dimethyl-1H-pyrazole-1-carbodithioate, can be performed according to the publication: “Bis(3,5-dimethylpyrazole-1-carbodithioato) Nickel(II) and Its Transformation to a Dinuclear Complex: Crystal Structure of [Ni2(í-3,5 Me2Pz)2(L1)2](L1) 3,5-dimethylpyrazole-1-carbodithioate)”. Inorg. Chem. 2001, 40, 1057.

Reagents and conditions that may be used for preparing a RAFT agent of formula (I) include: temperature in the range of 0° C. to ambient; solvents—alcohols, acetone, acetonitrile, dioxane, tetrahydrofuran, dimethylformamide (DMF), dimethylsulfoxide (DMSO); base—sodium hydroxide, potassium hydroxide, and sodium hydride. One set of conditions include using potassium hydroxide as the base in tetrahydrofuran at ambient temperature. The general procedure comprises starting with potassium hydroxide suspended in tetrahydrofuran in approximately a 0.5-1.0 M concentration at 4° C. The solution is then treated with approximately 1 equivalent of 3,5-dimethylpyrazole and followed by addition of approximately 1 equivalent of carbon disulfide. The resulting solution is then stirred (for example, for approximately 1 hour at ambient temperature) and the resulting potassium carbodithioate filtered and dried. The potassium carbodithioate was then dissolved in water before addition of approximately 1 equivalent of cyanomethyl halide (e.g. Cl, Br, I). Work-up may comprise, extraction with organic solvent, filtering, and drying. The desired RAFT agent may be purified by chromatography and/or re-crystallization and may be characterized by ¹H NMR, ¹³C NMR, and GC/MS.

RAFT agents in accordance with the invention are used in the method of preparing polymer, the method comprising polymerising under the control of the RAFT agent one or more ethylenically unsaturated monomers of formula (IV) and/or (V).

Monomers used in accordance with the invention are of formula (IV) or (V):

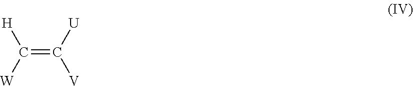

where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain,

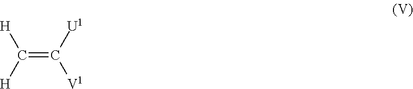

where U¹ is selected from H, C₁-C₄ alkyl or halogen; V¹ is halogen or of the form O-G where G is selected from —C(O)R¹ and —R¹, or V¹ is of the form NGG^{a} where G is as defined above and G^{a} is selected from H and R¹, G and G^{a} form together with N a heterocyclic ring, or V¹ is of the form CH₂G^{b} where G^{b} is selected from H, R¹, OH, OR¹, NR¹₂, PR¹₂, P(O)R¹₂, P(OR¹)₂, SR¹, SOR¹, and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

The or each R¹ in formula (IV) or (V) may be independently selected from optionally substituted C₁-C₂₂ alkyl, optionally substituted C₂-C₂₂ alkenyl, optionally substituted C₂-C₂₂ alkynyl, optionally substituted C₆-C₁₈ aryl, optionally substituted C₃-C₁₈ heteroaryl, optionally substituted C₃-C₁₈ carbocyclyl, optionally substituted C₂-C₁₈ heterocyclyl, optionally substituted C₇-C₂₄ arylalkyl, optionally substituted C₄-C₁₈ heteroarylalkyl, optionally substituted C₇-C₂₄ alkylaryl, optionally substituted C₄-C₁₈ alkylheteroaryl, and an optionally substituted polymer chain.

In one embodiment, R¹ in formula (IV) or (V) may be independently selected from optionally substituted C₁-C₄ alkyl.

Examples of optional substituents for R¹ in formula (IV) or (V) include those selected from alkyleneoxidyl (epoxy), hydroxy, alkoxy, acyl, acyloxy, formyl, alkylcarbonyl, carboxy, sulfonic acid, alkoxy- or aryloxy-carbonyl, isocyanato, cyano, silyl, halo, amino, including salts and derivatives thereof. Exemplar polymer chains include those selected from polyalkylene oxide, polyarylene ether and polyalkylene ether.

In the context of RAFT polymerisation, those skilled in the art will appreciate that monomers of formula (IV) and (V) are considered to have disparate reactivates. Monomers of formula (V) are generally considered to be less active toward RAFT polymerisation in that during polymerisation the carbon atom bearing the unpaired electron is attached to a sp³ hybridised carbon, oxygen, nitrogen, or halogen atom and therefore provides for a relatively unstabilised propagating radical. Monomers of formula (IV) are generally considered to be more active toward RAFT polymerisation in that during polymerisation the carbon atom bearing the unpaired electron is attached to a sp or sp² hybridised carbon atom that forms part of a double or triple bond, or are attached to a phosphorous or sulphur atom, and therefore provides for a relatively stabilised propagating radical.

Reference herein to the ethylenically unsaturated monomers used in accordance with the invention having “disparate reactivities” is intended to relate to the relative reactivities of the monomers in the context of RAFT polymerisation.

Examples of “more activated” monomers (i.e. monomers of formula (IV)) include acrylates, methacrylates, styrenics, vinyl aromatics and heteroaromatics, conjugated dienes, acrylamides, methacrylamides, acrylonitrile, methacrylonitrile, maleic anhydride and maleimides, vinyl sulphones, vinyl sulphoxides, vinyl phosphinates, vinyl phosphonates, and combinations thereof.

Specific examples of “more activated” monomers (i.e. monomers of formula (IV)) include methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacrylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, butadiene, chloroprene, acenapthalene, vinylnapthalene, vinylbiphenyl, vinyl azlactone; 1-vinylimidazole; 2-vinylpyridine, 4-vinyl pyridine, α-methylene-γ-butyrolactone, 2-methacryloxyethyl glucoside (any anomer), and vinylferrocene.

Examples of “less activated” monomers (i.e. monomers of formula (V)) include vinylethers, vinyl alkanoates, vinyl halides, N-vinyl amides, N-vinyl lactams, N-vinyl heteroarmoatics, vinyl silanes, vinyl phosphates and allyl or diallyl monomers.

Specific examples of “less activated” monomers (i.e. monomers of formula (V)) include vinyl acetate, vinyl propionate; vinyl butyrate, vinyl decanoate, vinyl neodecanoate, vinyl stearate; vinyl trifluoroacetate; vinyl benzoate, vinylester-based glycomonomers, ethyl vinyl ether, vinyl chloride, vinyl fluoride, vinyl bromide, N-vinylformamide, N-vinyl-N-methylacetamide, N-vinylphthalimide, N-vinylpyrrolidone, N-vinylcaprolactam, N-vinylcarbazole, vinyl trimethylsilane, vinyltriphenylsilane, vinyltrimethoxysilane, vinyltriethoxysilane, and diallyldimethylammonium chloride.

Where monomers of formula (IV) are polymerised in accordance with the invention, the monomers used may be the same so as to provide for a homopolymer or two or more different such monomers of formula (IV) may be used so as to provide for a copolymer.

Where monomers of formula (V) are polymerised in accordance with the invention, the monomers used may be the same so as to provide for a homopolymer, or two or more different monomers of formula (IV) may be used so as to provide for a copolymer.

A mixture of monomers of formula (IV) and (V) may also be polymerised in accordance with the invention.

Where the method of forming polymer in accordance with the invention involves polymerising different monomers of formula (IV), different monomers of formula (V), or a combination of monomers of formula (IV) and (V), those skilled in the art will appreciate that the resulting polymer may be referred to as a copolymer.

A copolymer may be in the form of a block copolymer, gradient copolymer or a random or statistical copolymer.

In one embodiment, polymer is prepared in accordance with the invention by polymerising under the control of the RAFT agent (I) a mixture of monomers of formula (IV) and (V) that is higher in concentration (e.g. comprises at least 60 mol %, or at least 70 mol %, or at least 80 mol %, or at least 90 mol %, or at least 95 mol %) of monomer of formula (V), relative to monomer of formula (IV).

In another embodiment, polymer is prepared in accordance with the invention by polymerising under the control of RAFT agent (I) a mixture of monomers of formula (IV) and (V) that is higher in concentration (e.g. comprises at least 60 mol %, or at least 70 mol %, or at least 80 mol %, or at least 90 mol %, or at least 95 mol %) of monomer of formula (IV), relative to monomer of formula (V).

Where a mixture of monomers of formula (IV) and (V) is used, it will be appreciated mol % of one monomer “relative” to the other will provide for a total of 100 mol %. For example, where the monomer mixture comprises 60 mol % monomer of formula (IV), that mol % “relative to monomer of formula (V) means the mixture will comprise 40 mol % of monomer of formula (V).

Factors that determine the copolymerisability of various monomers are well documented in the art. For example, see: Greenlee, R. Z., in Polymer Handbook 3^{rd} Edition (Brandup, J., and Immergut. E.H. Eds) Wiley: New York, 1989 p II/53.

A further summary of monomers amenable to RAFT polymerization can be found in recent reviews such as Moad et al, Polymer 49 (2008), 1079-1131.

A particular advantage afforded by RAFT polymerisation is the ability to produce polymer having a well defined molecular architecture, a predetermined molecular weight and a narrow molecular weight distribution or low dispersity (1)).

RAFT agents in accordance with the invention can advantageously provide for polymer having a low dispersity ( [symbol] ).

In one embodiment, polymer produced in accordance with a method of the invention, or polymer according to the invention, has a dispersity ( [symbol] ) of less than 1.7, or less than 1.6, or less than 1.5, or less than 1.4, or less than 1.3, or less than 1.2, or less than 1.1.

Polymer produced in accordance with a method of the invention, or polymer according to the invention, can advantageously have a dispersity ( [symbol] ) of less than 1.4, or less than 1.3, or less than 1.2, or less than 1.1.

As used herein, the dispersity ( [symbol] ) of polymer is determined according to equation (1):

[symbol]

=M_{w}/Mₙ (I)
where M_{w} is the mass average molecular weight, and Mₙ is the number average molecular weight.

M_{w} and Mₙ, referred to herein are intended to be that determined by Size Exclusion Chromatography (SEC) using poly(methyl methacrylate) standards.

RAFT agents in accordance with the invention have been found to be particularly well suited at polymerising monomers of formula (IV) and (V) that provide for a secondary incipient radical. By an ethylenically unsaturated monomer providing for a “secondary incipient radical” is meant that a secondary radical is produced by the monomer upon the ethylenically unsaturated functional group undergoing a free radical addition reaction.

In one embodiment, U of formula (IV) is H, thereby providing for formula (VI).

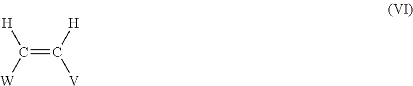

where W is H or forms together with V a lactone, anhydride or imide ring; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

In another embodiment, U¹ in formula (V) is H, thereby providing for formula (VII)

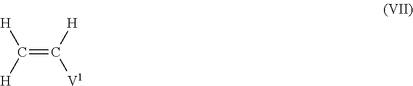

where V¹ is halogen or of the form O-G where G is selected from —C(O)R¹ and —R¹, or V¹ is of the form NGG^{a} where G is as defined above and G^{a} is selected from H and R¹, G and G^{a} form together with N a heterocyclic ring, or V¹ is of the form CH₂G^{b} where G^{b} is selected from H, R¹, OH, OR¹, NR¹₂, PR¹₂, P(O)R¹₂, P(OR¹)₂, SR¹, SOR¹, and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

Those skilled in the art will appreciate that monomers of formula (VI) fall within the scope of monomers of formula (IV), and that monomers of formula (VII) fall within the scope of monomers of formula (V). For convenience, reference herein to monomers of formula (IV) and (V) is also intended to be a reference to monomers of formula (VI) and (VII), respectively.

Examples of ethylenically unsaturated monomers of formula (IV) that provide for secondary incipient radicals and fall within the scope of formula (VI) include acrylates, styrenics, vinyl aromatics and heteroaromatics, conjugated dienes, acrylamides, acrylonitrile, maleic anhydride, maleimides, vinyl sulphones, vinyl sulphoxides, vinyl phosphinates, and vinyl phosphonates.

Specific examples of ethylenically unsaturated monomers of formula (IV) that provide for secondary incipient radicals and fall within the scope of formula (VI) include methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, N-methylolacrylamide, N-ethylolacrylamide, vinyl benzoic acid (all isomers), diethylamino styrene (all isomers), p-vinylbenzene sulfonic acid, p-vinylbenzene sulfonic sodium salt, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, butadiene, chloroprene, acenapthalene, vinylnapthalene, vinylbiphenyl, vinyl azlactone; 1-vinylimidazole; 2-vinylpyridine, 4-vinyl pyridine, and vinylferrocene.

Examples of ethylenically unsaturated monomers of formula (V) that provide for a secondary incipient radicals and fall within the scope of formula (VII) include vinylethers, vinyl alkanoates, vinyl halides, N-vinyl amides, N-vinyl lactams, N-vinyl heteroarmoatics, vinyl silanes, vinyl phosphates and allyl or diallyl monomers.

Specific examples of ethylenically unsaturated monomers of formula (V) that provide for secondary incipient radicals and fall within the scope of formula (VII) include vinyl acetate, vinyl propionate; vinyl butyrate, vinyl decanoate, vinyl neodecanoate, vinyl stearate; vinyl trifluoroacetate; vinyl benzoate, vinylester-based glycomonomers, ethyl vinyl ether, vinyl chloride, vinyl fluoride, vinyl bromide, N-vinylformamide, N-vinyl-N-methylacetamide, N-vinylphthalimide, N-vinylpyrrolidone, N-vinylcaprolactam, N-vinylcarbazole, vinyl trimethylsilane, vinyltriphenylsilane, vinyltrimethoxysilane, vinyltriethoxysilane, and diallyldimethylammonium chloride.

A notable feature of the present invention is that a RAFT agent of formula (I) can not only be used to polymerise monomers of formula (IV) and (V), but it can be used to prepare block copolymer that comprises a polymer block derived from one or more monomers of formula (IV) and a polymer block derived from one or more monomers of formula (V). In other words, the method of the invention may be used to prepare block copolymer comprising a LAM polymer block and a MAM polymer block.

A method of preparing block copolymer according to the invention comprises a first step (i) of polymerising under the control of a RAFT agent formula (I) one or more ethylenically unsaturated monomers of formula (IV) or (V) to form a macro-RAFT agent.

As used herein, the expression “macro-RAFT agent” is intended to mean a RAFT agent of formula (I) that has undergone reaction with monomer of formula (IV) or (V) so as to insert by the RAFT mechanism one or more monomer residue units.

According to the method of preparing block copolymer, a second step (ii) is performed in which one or more ethylenically unsaturated monomers of formula (IV) or (V) are polymerised under the control of the macro-RAFT agent formed in step (i). The monomer of formula (IV) or (V) polymerised in step (ii) is not of the same formula of monomer polymerised in step (i). Accordingly, step (i) provides for one polymer block derived from monomer of formula (IV) or (V), and step (ii) provides for a polymer block derived from monomer that is not of the same formula of monomer polymerised in step (i), resulting in formation of the block copolymer.

In one embodiment, block copolymers produced in accordance with the invention have a dispersity ( [symbol] ) of less than 1.4, or less than 1.3, or less than 1.2, or less than 1.1.

In another embodiment, block copolymers produced in accordance with the invention are derived from monomers of formula (IV) and monomers of formula (V) that provide for a secondary incipient radical.

In a further embodiment, step (i) of preparing block copolymer in accordance with the invention is performed using one or more ethylenically unsaturated monomers of formula (VI) or (VII), and step (ii) of that method is performed using one or more ethylenically unsaturated monomers of formula (VI) or (VII).

In one embodiment, the methods of preparing polymer, including block copolymer, in accordance with the invention is performed using monomer of formula (IV) selected from styrenes, acrylates, acrylamides, methacrylates and methacrylamides.

In a further embodiment, the methods for preparing polymer, including block copolymer, in accordance with the invention are performed using monomer of formula (V) selected from vinyl esters, vinyl ethers, vinyl sulphonates and vinyl amides.

Those skilled in the art will appreciate that polymer produced in accordance with the invention will have covalently bound thereto reaction residue of the RAFT agent of formula (I).

According to a method of preparing block copolymer in accordance with the invention, the block copolymer comprises a polymer block derived from one or more ethylenically unsaturated monomers of formula (IV) and a polymer block derived from one or more ethylenically unsaturated monomers of formula (V). The so formed block copolymer may therefore be described as comprising a *(PMAM)-(PLAM) diblock component, or a *(PLAM)-(PMAM) diblock component, where PMAM is a polymer block component derived from one or more ethylenically unsaturated monomers of formula (IV), PLAM is a polymer block component derived from one or more ethylenically unsaturated monomers of formula (V), and * denotes the point of covalent coupling of the diblock component that is closest to a sulfur atom of the reaction residue of the RAFT agent.

PMAM and PLAM are intended to represent polymer made up of RAFT reaction monomer residue units derived from one or more ethylenically unsaturated monomers selected from formula (IV) and (V), respectively. In other words, PMAM and PLAM represent polymer made up of RAFT polymerised MAM and RAFT polymerised LAM, respectively.

In one embodiment, a block copolymer (produced) in accordance with the invention comprises a *(PMAM)-(PLAM) diblock component selected from *(polydimethylacrylamide)-(polyvinyl acetate), *(polymethyl acrylate)-(polyvinyl acetate), *(polystyrene)-(polyvinyl acetate), *(polyacrylic acid)-(polyvinyl acetate), and *(polydimethylacrylamide)-(polyN-vinyl-pyrrolidone), where * denotes the point of covalent coupling of the diblock component that is closest to a sulfur atom of the reaction residue of the RAFT agent (or in formula (II)).

In another embodiment, a block copolymer (produced) in accordance with the invention comprises a *(PLAM)-(PMAM) diblock component selected from *(polyvinyl acetate)-(polydimethylacrylamide), *(polyvinyl acetate)-(polymethyl acrylate), *(polyvinyl acetate)-(polystyrene), *(polyvinyl acetate)-(polyacrylic acid) and *(polyN-vinyl-pyrrolidone)-(polydimethylacrylamide), where * denotes the point of covalent coupling of the diblock component that is closest to a sulfur atom of the reaction residue of the RAFT agent (or in formula (III)).

When preparing a block copolymer in accordance with the invention comprising a *(PMAM)-(PLAM) or *(PLAM)-(PMAM) diblock component it may be of assistance to precede step (i) of the method by reacting under the control of the RAFT agent of formula (I) one or more ethylenically unsaturated monomers of formula (IV) or (V) to form a macro-RAFT agent which is then used as the RAFT agent in step (i), wherein monomer reacted in this step preceding step (i) is not of the same formula of monomer polymerised in step (i).

For example, if step (i) is performed by polymerising monomer of formula (IV), then step (ii) of the method will be performed by polymerising monomer of formula (V). In that case, the step preceding step (i) will involve reacting monomer of formula (V).

Alternatively, step (i) may be performed by polymerising monomer of formula (V), thereby requiring step (ii) to proceed by polymerising monomer of formula (IV). In that case, the step preceding step (i) will involve reacting monomer of formula (IV).

Those skilled in the art will appreciate that where step (i) is preceded by reacting under the control of the RAFT agent of formula (I) one or more ethylenic ally unsaturated monomers of formula (IV) or (V) a macro-RAFT agent will be formed. In that case, the so formed macro-RAFT agent will be the RAFT agent used in step (i). Performing step (i) will therefore result in the formation of a new macro-RAFT agent, which in turn will be used in step (ii).

Where step (i) in the method of preparing block copolymer in accordance with the invention is preceded by reacting under the control of the RAFT agent of formula (I) one or more ethylenically unsaturated monomers of formula (IV) or (V), the resulting block copolymer may be described as comprising *(PMAM)-(PLAM)-(MAMi) or *(PLAM)-(PMAM) (LAMi) where PMAM and MAMi are as herein described, PLAM and LAMI are as herein described, and * denotes the point of covalent coupling closest to a sulfur atom of the RAFT agent reaction residue.

Where step (i) is preceded by reacting under the control of the RAFT agent of formula (I) one or more ethylenically unsaturated monomers of (IV) or (V), the so formed macro-RAFT agent will generally comprise from 1 to about 20, or from 1 to about 15, or from 1 to about 10 RAFT reaction monomer residue units derived from one or more ethylenically unsaturated monomers of formula (IV) or (V). In other words, the step preceding step (i) will generally be used to insert a relatively low number of monomer residue units into the RAFT agent.

In one embodiment, step (i) is preceded by reacting under the control of the RAFT agent of formula (I) one or more ethylenically unsaturated monomers of formula (IV).

In another embodiment, step (i) is preceded by reacting under the control of the RAFT agent of formula (I) one or more ethylenically unsaturated monomers of formula (V).

Without wishing to be limited by theory, it is believed preceding step (i) by inserting a relatively low number of monomer residue units into the RAFT agent can facilitate formation of certain block copolymers.

A preceding step of inserting a relatively low number of monomer residue units into a RAFT agent of formula (I) may also facilitate formation of other polymer structures in general.

For example, the method according to the invention of forming polymer comprises polymerising under the control of the RAFT agent one or more ethylenically unsaturated monomers of formula (IV) and/or (V). In one embodiment, this method may be preceded by reacting under the control of the RAFT agent of formula (I) one or more ethylenically unsaturated monomers of formula (IV) or (V) to form a macro-RAFT agent, wherein one or more ethylenically unsaturated monomers of formula (IV) or (V) are polymerised under the control of the so formed macro-RAFT agent to form the intended polymer, and wherein monomer reacted in the preceding step is not of the same formula of monomer polymerised in the step of preparing the intended polymer.

For example, if the step of preparing the intended polymer is performed by polymerising monomer of formula (IV), then the preceding step will involve reacting monomer of formula (V).

Alternatively, if the step of preparing the intended polymer is performed by polymerising monomer of formula (V), then the preceding step will involve reacting monomer of formula (IV).

The preceding step may also comprise a monomer mixture that is has a higher concentration of monomer that is not of the monomer formula that is polymerised to form the intended polymer.

For example, if the step of preparing the intended polymer is performed by polymerising monomer of formula (IV), then the preceding step may involve reacting a mixture of monomers of formula (IV) and (V) that is higher in concentration (e.g. at least 60 mol %, or at least 70 mol %, or at least 80 mol %, or at least 90 mol %, or at least 95 mol %) of monomer of formula (V), relative to monomer of formula (IV).

Alternatively, if the step of preparing the intended polymer is performed by polymerising monomer of formula (V), then the preceding step may involve reacting a mixture of monomers of formula (IV) and (V) that is higher in concentration (e.g. at least 60 mol %, or at least 70 mol %, or at least 80 mol %, or at least 90 mol %, or at least 95 mol %) of monomer of formula (IV), relative to monomer of formula (V).

In one embodiment, the method according to the invention may therefore comprises reacting under the control of the RAFT agent of formula (I) one or more ethylenically unsaturated monomers of formula (IV), or a mixture of monomers of formula (IV) and (V), to form a macro-RAFT, and then polymerising one or more ethylenically unsaturated monomers of formula (V) under the control of the so form macro-RAFT agent.

In another embodiment, the method according to the invention may therefore comprises reacting under the control of the RAFT agent of formula (I) one or more ethylenically unsaturated monomers of formula (V), or a mixture of monomers of formula (IV) and (V), to form a macro-RAFT, and then polymerising one or more ethylenically unsaturated monomers of formula (IV) under the control of the so formed macro-RAFT agent.

The methods of preparing polymer in accordance with the present invention can advantageously be performed using techniques and reagents well known to those skilled in the art. In addition to functioning as a true all purpose RAFT agent, RAFT agents in accordance with the invention advantageously can be used to form polymer in a similar manner to conventional RAFT agents.

Polymerisation of the monomers will usually require initiation from a source of free radicals. The source of initiating radicals can be provided by any suitable method of generating free radicals, such as the thermally induced homolytic scission of suitable compound(s) (thermal initiators such as peroxides, peroxyesters, or azo compounds), the spontaneous generation from monomers (e.g. styrene), redox initiating systems, photochemical initiating systems or high energy radiation such as electron beam, X- or gamma-radiation. The initiating system is chosen such that under the reaction conditions there is no substantial adverse interaction of the initiator or the initiating radicals with the RAFT agent under the conditions of the reaction. The initiator ideally should also have the requisite solubility in the reaction medium.

Thermal initiators are chosen to have an appropriate half life at the temperature of polymerisation. These initiators can include one or more of the following compounds:
2,2′-azobis(isobutyronitrile), 2,2′-azobis(2-cyanobutane), dimethyl 2,2′-azobis(isobutyrate), 4,4′-azobis(4-cyanovaleric acid), 1,1′-azobis(cyclohexanecarbonitrile), 2-(t-butylazo)-2-cyanopropane, 2,2′-azobis{2-methyl-N-[1,1-bis(hydroxymethyl)-2-hydroxyethyl]propionamide}, 2,2′-azobis[2-methyl-N-(2-hydroxyethyl)propionamide], 2,2′-azobis(N,N′-dimethyleneisobutyramidine) dihydrochloride, 2,2′-azobis(2-amidinopropane) dihydrochloride, 2,2′-azobis(N,N′-dimethyleneisobutyramidine), 2,2′-azobis{2-methyl-N-[1,1-bis(hydroxymethyl)-2-hydroxyethyl]propionamide}, 2,2′-azobis{2-methyl-N-[1,1-bis(hydroxymethyl)-2-ethyl]propionamide}, 2,2′-azobis[2-methyl-N-(2-hydroxyethyl)propionamide], 2,2′-azobis(isobutyramide) dihydrate, 2,2′-azobis(2,2,4-trimethylpentane), 2,2′-azobis(2,4-dimethyl-4-methoxyvaleronitrile), 2,2′-azobis(2-methylpropane), t-butyl peroxyacetate, t-butyl peroxybenzoate, t-butyl peroxyneodecanoate, t-butylperoxy isobutyrate, t-amyl peroxypivalate, t-butyl peroxypivalate, diisopropyl peroxydicarbonate, dicyclohexyl peroxydicarbonate, dicumyl peroxide, dibenzoyl peroxide, dilauroyl peroxide, potassium peroxydisulfate, ammonium peroxydisulfate, di-t-butyl hyponitrite, dicumyl hyponitrite. This list is not exhaustive.

Photochemical initiator systems are chosen to have the requisite solubility in the reaction medium and have an appropriate quantum yield for radical production under the conditions of the polymerisation. Examples include benzoin derivatives, benzophenone, acyl phosphine oxides, and photo-redox systems.

Redox initiator systems are chosen to have the requisite solubility in the reaction medium and have an appropriate rate of radical production under the conditions of the polymerisation; these initiating systems can include, but are not limited to, combinations of the following oxidants and reductants:
oxidants: potassium, peroxydisulfate, hydrogen peroxide, t-butyl hydroperoxide.
reductants: iron (II), titanium (III), potassium thiosulfite, potassium bisulfite.

Other suitable initiating systems are described in recent texts. See, for example, Moad and Solomon “the Chemistry of Free Radical Polymerisation”, Pergamon, London, 1995, pp 53-95.

Reaction conditions for the polymerisation should be chosen such that the ratio of the total number of initiator-derived radicals to the number of RAFT agent molecules is maintained at a minimum value consistent with achieving an acceptable rate of polymerisation. Generally, such a ratio is less than 1:1, or less than 1:10, or in the range of 1:10 to 1:5000.

Bearing the above consideration in mind, the initiator concentration will be chosen so as to give an acceptable rate of polymerization of the specific monomer or monomer combination.

Those skilled in the art will appreciate that in the application of RAFT agents the chain transfer constant is considered an important parameter of the addition-fragmentation steps that occur in the polymerisation process. A consideration of chain transfer constants for RAFT agents is given in WO 98/01478.

The methods of the invention may be carried out using solution, emulsion, bulk or suspension polymerisation techniques in either batch, semi-batch, continuous, or feed modes.

For heterogeneous polymerisation, it is desirable to choose a RAFT agent which has appropriate solubility parameters. For aqueous emulsion polymerisation reactions, the RAFT agent should preferably partition in favour of the organic (monomer) phase and yet have sufficient aqueous solubility that it is able to distribute between the monomer droplet phase and the polymerisation locus.

The choice of polymerisation conditions can be important. The reaction temperature may influence the rate parameters discussed above. For example, higher reaction temperatures can increase the rate of fragmentation. Conditions should be chosen such that the number of polymer chains formed from initiator-derived radicals is minimised to an extent consistent with obtaining an acceptable rate of polymerisation. Termination of polymerisation by radical-radical reaction will lead to chains which contain no active group and therefore cannot be reactivated. The rate of radical-radical termination is proportional to the square of the radical concentration. These reaction conditions may therefore require careful choice of the initiator concentration and, where appropriate the rate of the initiator feed.

It is also desirable to choose other components of the reaction medium (for example, the solvents, surfactants, additives, and initiator) such that they have a low transfer constant towards the propagating radical. Chain transfer to these species will lead to the formation of polymer chains which do not contain the active RAFT group.

As a general guide in choosing conditions for the synthesis of narrow polydispersity polymers, the concentration of initiator(s) and other reaction conditions (solvent(s) if any, reaction temperature, reaction pressure, surfactants if any, other additives) should be chosen such that the molecular weight of polymer formed in the absence of the RAFT agent is at least twice that formed in its presence. In polymerisations where termination is solely by disproportionation, this equates to choosing an initiator concentration such that the total moles of initiating radicals formed during the polymerisation is less than 0.5 times that of the total moles of RAFT agent. It can be desirable to choose conditions such that the molecular weight of polymer formed in the absence of the RAFT agent is at least 5-fold that formed in its presence ([initiating radicals]/[RAFT agent]<0.2).

Thus, the dispersity ( [symbol] ) can be controlled by varying the number of moles of RAFT agent to the number of moles initiating radicals. Lower dispersities ( [symbol] ) can be obtained by increasing this ratio; higher dispersities ( [symbol] ) can be obtained by decreasing this ratio.

Polymerisation will generally be carried out at temperatures in the range of −20 to 200° C., for example in the range of 40 to 160° C. The polymerisation temperature may be chosen taking into consideration the specific monomer(s) being polymerised and other components of the polymerisation or reaction medium.

In the case of emulsion or suspension polymerisation the reaction medium will often be predominantly water and conventional stabilisers, dispersants and other additives may also be present.

For solution polymerisation, the reaction medium can be chosen from a wide range of media to suit the monomer(s) being used. For example, water; alcohols, such as methanol, ethanol, 2-propanol and 2-butanol; aromatic hydrocarbons, such as toluene, xylenes or petroleum naphtha; ketones, such as methyl amyl ketone, methyl isobutyl ketone, methyl ethyl ketone or acetone; esters, such as butyl acetate or hexyl acetate; ethers, such as 1,2-dimethoxyethane, tetrahydrofuran and dioxane; and glycol ether esters, such as propylene glycol monomethyl ether acetate.

Polymer according to the invention, or that produced in accordance with the methods of the invention, is made up of RAFT reaction monomer residue units derived from (i) one or more ethylenically unsaturated monomers of formula (IV), (ii) one or more ethylenically unsaturated monomers of formula (V), or (iii) a combination of one or more ethylenically unsaturated monomers of formula (IV) and one or more ethylenically unsaturated monomers of formula (V). The so formed polymer may be in the form of a homopolymer, copolymer, block copolymer, multiblock copolymer, gradient copolymer, or random or statistical copolymer.

Polymer according to the invention is defined by formula (II) or (III)

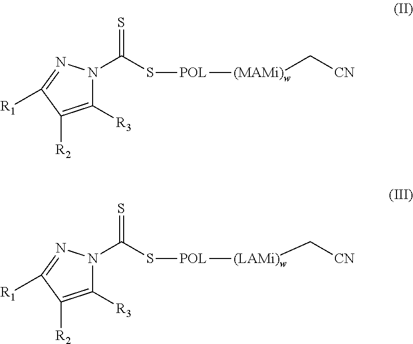

where R₁, R₂ and R₃ are each independently selected from H and optionally substituted alkyl (for example optionally substituted C₁-C₁₂ alkyl), POL in formula (II) is a polymer chain comprising (PMAM)ₓ-(PLAM)_{y}*, POL in formula (III) is a polymer chain comprising (PLAM)_{y}-(PMAM)ₓ*, MAMi and PMAM are each independently made up of one or more RAFT reaction monomer residue units derived from one or more ethylenically unsaturated monomers of formula (IV), LAMi and PLAM are each independently made up of one or more RAFT reaction monomer residue units derived from one or more ethylenically unsaturated monomers selected from formula (V), w, x and y are each independently 0 or 1 provided at least one of x or y is 1, and * denotes the point of covalent coupling to (i) MAMi or LAMi when present, or (ii) —CH₂—CN when MAMi or LAMi are not present.

Polymer of formula (II) or (III) comprises a polymer chain (POL). POL in formula (II) is a polymer chain comprising (PMAM)ₓ-(PLAM)_{y}*, and POL in formula (III) is a polymer chain comprising (PLAM)_{y}-(PMAM)ₓ*. The terms x and y are each independently 0 or 1, provided at least one of x or y is 1. As mentioned, PMAM and PLAM represent polymer made up of RAFT reaction monomer residue units derived from one or more ethylenically unsaturated monomers selected from formula (IV) and (V), respectively. In other words, PMAM and PLAM represent polymer made up of RAFT polymerised MAM and RAFT polymerised LAM, respectively.

Each PMAM and PLAM polymer chain may be a homopolymer or copolymer as herein described.

Where x and y are both 1, (PMAM)ₓ-(PLAM)_{y}* and (PLAM)_{y}-(PMAM)ₓ* may represent a polymer chain, or part thereof, comprising copolymer derived from monomer of formula (IV) and (V).

In addition to the (PMAM)ₓ-(PLAM)_{y}* or a (PLAM)_{y}-(PMAM)ₓ* polymer chain, POL in formula (II) or (III) may comprise additional PMAM and/or PLAM polymer chain components. For example, POL in formula (II) may be a polymer chain comprising (PLAM)-(PMAM)-(PLAM)-(PMAM)-(PLAM)*, and POL in formula (III) may be a polymer chain comprising (PMAM)-(PLAM)-(PMAM)-(PLAM)-(PMAM)-(PLAM)-(PMAM)*.

In formula (II) and (III) * denotes the point of covalent coupling to (i) MAMi or LAMi when present, or (ii) —CH₂—CN when MAMi or LAMi are not present. In other words, in formula (II) where PMAM, PLAM and MAMi are present they will be covalently coupled to provide for (MAM)-(LAM)-(MAMi). In formula (III) when PLAM, PMAM and LAMi are present they will be covalently coupled to provide for (PLAM)-(PMAM)-(LAMi).

For avoidance of any doubt, where y in formula (II) is 0, (PMAM)ₓ-(PLAM)_{y}* becomes (PMAM)ₓ*, and * denotes the point of covalent coupling to MAMi when present or —CH₂—CN when MAMi is not present. Similarly, in formula (III) when x is 0 (PLAM)_{y}-(PMAM)ₓ* becomes (PLAM)_{y}*, and * denotes the point of covalent coupling to LAMi when present or —CH₂—CN when LAMi is not present.

Generally, where y in formula (II) is 0, w is also 0. Similarly, when x in formula (III) is 0, w is also 0.

There is no particular limitation on the number of RAFT reaction monomer residue units that can make up the PMAM and PLAM polymer chain components of POL in formula (II) and (III). Generally, PMAM and PLAM in formula (II) and (III) will each independently represent at least about 10, or at least about 15, or at least about 20 RAFT reaction monomer residue units of one or more ethylenically unsaturated monomers of formula (IV) and (V), respectively.

When present, MAMi and LAMi in formula (II) and (III) will generally represent a relatively low number of RAFT reaction monomer residue units derived from one or more ethylenically unsaturated monomers of formula (IV) or (V), respectively.

For example, MAMi may represent from 1 to about 20, or from 1 to about 15, or from 1 to about 10 RAFT reaction monomer residue units derived from one or more ethylenically unsaturated monomers of formula (IV), and LAMi may represent from 1 to about 20, or from 1 to about 15, or from 1 to about 10 RAFT reaction monomer residue units derived from one or more ethylenically unsaturated monomers of formula (IV).

As previously mentioned, it may be desirable to include a MAMi or LAMi component in polymer according to the invention to facilitate the preparation of certain (PLAM)-(PMAM) or (PMAM)-(PLAM) block copolymers, or certain (PLAM) or (PMAM) polymers.

As used herein, the term “alkyl”, used either alone or in compound words denotes straight chain, branched or cyclic alkyl, preferably C₁₋₂₀ alkyl, e.g. C₁₋₁₀ or C₁₋₆ Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substituents as herein defined.

The term “alkenyl” as used herein denotes groups formed from straight chain, branched or cyclic hydrocarbon residues containing at least one carbon to carbon double bond including ethylenically mono-, di- or polyunsaturated alkyl or cycloalkyl groups as previously defined, preferably C₂₋₂₀ alkenyl (e.g. C₂₋₁₀ or C₂₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethylenically mono-, di- or polyunsaturated alkyl or cycloalkyl groups as previously defined. Unless the number of carbon atoms is specified the term preferably refers to C₂₋₂₀ alkynyl (e.g. C₂₋₁₀ or C₂₋₆). Examples include ethynyl, 1-propynyl, 2-propynyl, and butynyl isomers, and pentynyl isomers. An alkynyl group may be optionally substituted by one or more optional substituents as herein defined.

The term “halogen” (“halo”) denotes fluorine, chlorine, bromine or iodine (fluoro, chloro, bromo or iodo). Preferred halogens are chlorine, bromine or iodine.

The term “aryl” (or “carboaryl)” denotes any of single, polynuclear, conjugated and fused residues of aromatic hydrocarbon ring systems (e.g C₆₋₁₈ aryl). Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl and naphthyl. An aryl group may or may not be optionally substituted by one or more optional substituents as herein defined. The term “arylene” is intended to denote the divalent form of aryl.

The term “carbocyclyl” includes any of non-aromatic monocyclic, polycyclic, fused or conjugated hydrocarbon residues, preferably C₃₋₂₀ (e.g. C₃₋₁₀ or C₃₋₈). The rings may be saturated, e.g. cycloalkyl, or may possess one or more double bonds (cycloalkenyl) and/or one or more triple bonds (cycloalkynyl). Particularly preferred carbocyclyl moieties are 5-6-membered or 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl, indanyl, decalinyl and indenyl. A carbocyclyl group may be optionally substituted by one or more optional substituents as herein defined. The term “carbocyclylene” is intended to denote the divalent form of carbocyclyl.

The term “heterocyclyl” when used alone or in compound words includes any of monocyclic, polycyclic, fused or conjugated hydrocarbon residues, preferably C₃₋₂₀ (e.g. C₃₋₁₀ or C₃₋₈) wherein one or more carbon atoms are replaced by a heteroatom so as to provide a non-aromatic residue. Suitable heteroatoms include O, N, S, P and Se, particularly O, N and S. Where two or more carbon atoms are replaced, this may be by two or more of the same heteroatom or by different heteroatoms. The heterocyclyl group may be saturated or partially unsaturated, i.e. possess one or more double bonds. Particularly preferred heterocyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetrahydropyrrolyl, tetrahydrothiophenyl, pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, pyranyl and dihydropyranyl. A heterocyclyl group may be optionally substituted by one or more optional substituents as herein defined. The term “heterocyclylene” is intended to denote the divalent form of heterocyclyl.

The term “heteroaryl” includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadiazolyl, oxatriazolyl, triazinyl, and furazanyl. A heteroaryl group may be optionally substituted by one or more optional substituents as herein defined. The term “heteroarylene” is intended to denote the divalent form of heteroaryl.

The term “acyl” either alone or in compound words denotes a group containing the moiety C═O (and not being a carboxylic acid, ester or amide) Preferred acyl includes C(O)—R^{e}, wherein R^{e} is hydrogen or an alkyl, alkenyl, alkynyl, aryl, heteroaryl, carbocyclyl, or heterocyclyl residue. Examples of acyl include formyl, straight chain or branched alkanoyl (e.g. C₁₋₂₀) such as acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. The R^{x} residue may be optionally substituted as described herein.

The term “sulfoxide”, either alone or in a compound word, refers to a group —S(O)R^{f} wherein R^{f} is selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl.

The term “sulfonyl”, either alone or in a compound word, refers to a group S(O)₂—R^{f}, wherein R^{f} is selected from hydrogen, alkyl, alkenyl, alkynyl, aryl, heteroaryl, heterocyclyl, carbocyclyl and aralkyl. Examples of preferred R^{f} include C₁₋₂₀alkyl, phenyl and benzyl.

The term “sulfonamide”, either alone or in a compound word, refers to a group S(O)NR^{f}R^{f} wherein each R^{f} is independently selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl. In a preferred embodiment at least one R^{f} is hydrogen. In another form, both R^{f} are hydrogen.

The term, “amino” is used here in its broadest sense as understood in the art and includes groups of the formula NR^{a}R^{b} wherein R^{a} and R^{b} may be independently selected from hydrogen, alkyl, alkenyl, alkynyl, aryl, carbocyclyl, heteroaryl, heterocyclyl, arylalkyl, and acyl. R^{a} and R^{b}, together with the nitrogen to which they are attached, may also form a monocyclic, or polycyclic ring system e.g. a 3-10 membered ring, particularly, 5-6 and 9-10 membered systems. Examples of “amino” include NH₂, NHalkyl (e.g. C₁₋₂₀alkyl), NHaryl (e.g. NHphenyl), NHaralkyl (e.g. NHbenzyl), NHacyl (e.g. NHC(O)C₁₋₂₀alkyl, NHC(O)phenyl), Nalkylalkyl (wherein each alkyl, for example C₁₋₂₀, may be the same or different) and 5 or 6 membered rings, optionally containing one or more same or different heteroatoms (e.g. O, N and S).

The term “amido” is used here in its broadest sense as understood in the art and includes groups having the formula C(O)NR^{a}R^{b}, wherein R^{a} and R^{b} are as defined as above. Examples of amido include C(O)NH₂, C(O)NHalkyl (e.g. C₁₋₂₀alkyl), C(O)NHaryl (e.g. C(O)NHphenyl), C(O)NHaralkyl (e.g. C(O)NHbenzyl), C(O)NHacyl (e.g. C(O)NHC(O)C₁₋₂₀alkyl, C(O)NHC(O)phenyl), C(O)Nalkylalkyl (wherein each alkyl, for example C₁₋₂₀, may be the same or different) and 5 or 6 membered rings, optionally containing one or more same or different heteroatoms (e.g. O, N and S).

The term “carboxy ester” is used here in its broadest sense as understood in the art and includes groups having the formula CO₂R^{g}, wherein R^{g} may be selected from groups including alkyl, alkenyl, alkynyl, aryl, carbocyclyl, heteroaryl, heterocyclyl, aralkyl, and acyl. Examples of carboxy ester include CO₂C₁₋₂₀alkyl, CO₂aryl (e.g. CO₂phenyl), CO₂aralkyl (e.g. CO₂ benzyl).

In this specification “optionally substituted” is taken to mean that a group may or may not be substituted or fused (so as to form a condensed polycyclic group) with one, two, three or more of organic and inorganic groups, including those selected from: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl, nitrocarbocyclyl, nitroacyl, nitroaralkyl, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyl, cyano, sulfate and phosphate groups. Optional substitution may also be taken to refer to where a —CH₂— group in a chain or ring is replaced by a group selected from —O—, —S—, —NR^{a}—, —C(O)— (i.e. carbonyl), —C(O)O— (i.e. ester), and —C(O)NR^{a}— (i.e. amide), where R^{a} is as defined herein.

Preferred optional substituents include alkyl, (e.g. C₁₋₆ alkyl such as methyl, ethyl, propyl, butyl, cyclopropyl, cyclobutyl, cyclopentyl or cyclohexyl), hydroxyalkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc) alkoxy (e.g. C₁₋₆ alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆ alkyl, halo, hydroxy, hydroxyC₁₋₆ alkyl, C₁₋₆ alkoxy, haloC₁₋₆alkyl, cyano, nitro OC(O)C₁₋₆ alkyl, and amino), benzyl (wherein benzyl itself may be further substituted e.g., by C₁₋₆ alkyl, halo, hydroxy, hydroxyC₁₋₆alkyl, C₁₋₆ alkoxy, haloC₁₋₆ alkyl, cyano, nitro OC(O)C₁₋₆ alkyl, and amino), phenoxy (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), benzyloxy (wherein benzyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), amino, alkylamino (e.g. C₁₋₆ alkyl, such as methylamino, ethylamino, propylamino etc), dialkylamino (e.g. C₁₋₆ alkyl, such as dimethylamino, diethylamino, dipropylamino), acylamino (e.g. NHC(O)CH₃), phenylamino (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), nitro, formyl, —C(O)-alkyl (e.g. C₁₋₆ alkyl, such as acetyl), O—C(O)-alkyl (e.g. C₁₋₆alkyl, such as acetyloxy), benzoyl (wherein the phenyl group itself may be further substituted e.g., by C₁₋₆ alkyl, halo, hydroxy hydroxyC₁₋₆ alkyl, C₁₋₆ alkoxy, haloC₁₋₆ alkyl, cyano, nitro OC(O)C₁₋₆alkyl, and amino), replacement of CH₂ with C═O, CO₂H, CO₂alkyl (e.g. C₁₋₆ alkyl such as methyl ester, ethyl ester, propyl ester, butyl ester), CO₂phenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONH₂, CONHphenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONHbenzyl (wherein benzyl itself may be further substituted e.g., by C₁₋₆ alkyl, halo, hydroxy hydroxyl C₁₋₆ alkyl, C₁₋₆ alkoxy, halo C₁₋₆ alkyl, cyano, nitro OC(O)C₁₋₆ alkyl, and amino), CONHalkyl (e.g. C₁₋₆ alkyl such as methyl ester, ethyl ester, propyl ester, butyl amide) CONHdialkyl (e.g. C₁₋₆ alkyl) aminoalkyl (e.g., HNC₁₋₆ alkyl-, C₁₋₆alkylHN—C₁₋₆ alkyl- and (C₁₋₆ alkyl)₂N—C₁₋₆ alkyl-), thioalkyl (e.g., HS C₁₋₆ alkyl-), carboxyalkyl (e.g., HO₂CC₁₋₆ alkyl-), carboxyesteralkyl (e.g., C₁₋₆ alkylO₂CC₁₋₆ alkyl-), amidoalkyl (e.g., H₂N(O)CC₁₋₆ alkyl-, H(C₁₋₆ alkyl)N(O)CC₁₋₆ alkyl-), formylalkyl (e.g., OHCC₁₋₆alkyl-), acylalkyl (e.g., C₁₋₆ alkyl(O)CC₁₋₆ alkyl-), nitroalkyl (e.g., O₂NC₁₋₆ alkyl-), sulfoxidealkyl (e.g., R(O)SC₁₋₆ alkyl, such as C₁₋₆ alkyl(O)SC₁₋₆ alkyl-), sulfonylalkyl (e.g., R(O)₂SC₁₋₆ alkyl-such as C₁₋₆ alkyl(O)₂SC₁₋₆ alkyl-), sulfonamidoalkyl (e.g., ₂HRN(O)SC₁₋₆ alkyl, H(C₁₋₆ alkyl)N(O)SC₁₋₆ alkyl-).

The term “heteroatom” or “hetero” as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

For monovalent substituents, terms written as “[groupA][group B]” refer to group A when linked by a divalent form of group B. For example, “[group A][alkyl]” refers to a particular group A (such as hydroxy, amino, etc.) when linked by divalent alkyl, i.e. alkylene (e.g. hydroxyethyl is intended to denote HO—CH₂—CH—). Thus, terms written as “[group]oxy” refer to a particular group when linked by oxygen, for example, the terms “alkoxy” or “alkyloxy”, “alkenoxy” or “alkenyloxy”, “alkynoxy” or alkynyloxy”, “aryloxy” and “acyloxy”, respectively, denote alkyl, alkenyl, alkynyl, aryl and acyl groups as hereinbefore defined when linked by oxygen. Similarly, terms written as “[group]thio” refer to a particular group when linked by sulfur, for example, the terms “alkylthio”, “alkenylthio”, alkynylthio” and “arylthio”, respectively, denote alkyl, alkenyl, alkynyl and aryl groups as hereinbefore defined when linked by sulfur.

The invention will hereinafter be described with reference to the following non-limiting examples.

### EXAMPLES

### General

Number (Mₙ) and weight (M_{w}) average molecular weight of the polymers and their dispersity ( [symbol] ) were measured using size exclusion chromatography (SEC). SEC was performed on a Shimadzu system equipped with a CMB-20A controller system, a SIL-20A HT autosampler, a LC-20AT tandem pump system, a DGU-20A degasser unit, a CTO-20AC column oven, a RDI-10A refractive index (RI) detector, and a Styragel HT (Waters) four column set (HT2, HT3, HT4 and HT5). N,N-dimethylacetamide (DMAc) (containing 4.3 g·l LiBr⁻¹) was used as eluent at a flow rate of 1 ml/min (pressure range: 750-800 psi). The column temperature was set to 80° C. and the temperature at the RI detector was set to 40° C. The SEC was calibrated with narrow dispersity poly(methyl methacrylate) standards, and molecular weights are reported as poly(methyl methacrylate) equivalents. Mₙ and [symbol] were evaluated using Shimadzu software (LabSolutions version 5.63). A 3^{rd} order polynomial was used to fit the log M vs. time calibration curve, which was near linear across the molecular weight ranges.

### RAFT Agent Preparation

### Example 1

### Potassium 3,5-dimethyl-1H-pyrazole-1-carbodithioate (1)

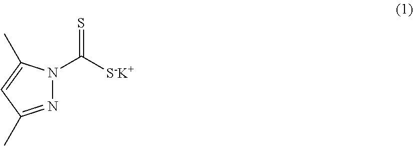

Prepared according to that described in Inorg. Chem. 2001, 40, 1057. To a solution of potassium hydroxide (6.16 g, 109.75 mmol, 1.06 equivalents) in tetrahydrofuran (100 mL) at 4° C. was added N, N-dimethylpyrazole (10.00 g, 104.03 mmol). The mixture was stirred at 4° C. for 5 minutes and carbon disulfide (8.17 mL, 10.30 g, 135.23 mmol, 1.3 equivalents) was then added slowly. The reaction mixture was stirred at 4° C. for 3 minutes and then at room temperature for 50 minutes. The mixture was filtered and the orange solid washed with diethyl ether and dried to yield (1) as a light orange solid (19.52 g, 89%). NMR data according to literature.

### Cyanomethyl 3,5-dimethyl-1H pyrazole-1-carbodithioate (2)

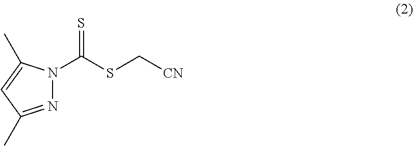

Potassium 3,5-dimethyl-1H-pyrazole-1-carbodithioate (1) (18.60 g, 88.42 mmol) was dissolved in deionised water (350 mL) and then cooled in an ice-water bath. Chloroacetonitrile (5.92 mL, 7.01 g, 92.85 mmol, 1.05 equivalents) was then added and the reaction mixture was stirred in the ice-water bath for 2 minutes, and then at room temperature for one hour and forty minutes. The mixture was then cooled down in an ice-water bath for fifteen minutes and filtered. The yellow solid was collected, washed with ice-cold water, and then dried to give a first crop of product. The mother liquor was then stirred for 2 days, filtered and the yellow solid washed with ice-cold water and dried to give a second crop of product. The two crops were combined to give the product as a pale yellow solid (2) (11.49 g, 61%). ¹H NMR (CDCl₃) δ (ppm) 2.26 (s, CH₃), 2.68 (d, 2H, CH₃), 3.95 (s, 2H, CH₂CN); 6.12 (d, 1H, ArH). ¹³C NMR (CDCl₃) δ (ppm) 13.8, 17.2, 21.7, 114.2, 115.5, 146.3, 153.1, 195.4.

### Polymer Preparation

In all instances, monomers were purified (to remove inhibitors) and flash-distilled immediately prior to use. The experiments referred to as “reference examples” were experiments run with RAFT agents, currently commercially available and in common usage. For polymerizations performed in ampoules, degassing was accomplished by repeated freeze-evacuate-thaw cycles. Once degassing was complete, the ampoules were flame sealed under vacuum and completely submerged in an oil bath at the specified temperature for the specified times. The percentage conversions were calculated gravimetrically unless otherwise indicated. Examples 2 to 6 are examples of making polymers with less activated monomers.

### Example 2

### Preparation of poly(N,N-dimethyl acrylamide) using cyanomethyl 3,5-dimethyl-1H pyrazole-1-carbodithioate (1) at 100° C.

A solution containing N,N-dimethylacrylamide (0.618 mL), 1,1′-azobiscyanocyclohexane (2.93 mg), cyanomethyl 3,5-dimethyl-1H pyrazole-1-carbodithioate (1) (12.68 mg, 0.03M), and acetonitrile (1.382 mL) was prepared. The resulting mixture was degassed, sealed and heated at 100° C. for 1 hour. The volatiles were removed in vacuo to give poly(dimethyl acrylamide) at 99% conversion (based on the consumption of dimethyl acrylamide), with Mₙ 15,376, M_{w}/Mₙ 1.066.

### Example 3

### Preparation of poly(methyl acrylate) using cyanomethyl 3,5-dimethyl-1H pyrazole-1-carbodithioate (1) at 100° C.

A solution containing methyl acrylate (0.540 mL), 1,1′-azobiscyanocyclohexane (2.93 mg), cyanomethyl 3,5-dimethyl-1H pyrazole-1-carbodithioate (1) (12.68 mg, 0.03M), and acetonitrile (1.460 mL) was prepared. The resulting mixture was degassed, sealed and heated at 100° C. for 1 hour. The volatiles were removed in vacuo to give poly(methyl acrylate) at 77% conversion (based on the consumption of methyl acrylate), with Mₙ 9,735, M_{w}/Mₙ 1.109.

### Example 4

### Preparation of poly(acrylic acid) using cyanomethyl 3,5-dimethyl-1H pyrazole-1-carbodithioate (1) at 100° C.

A solution containing acrylic acid (0.411 mL), 1,1′-azobiscyanocyclohexane (2.93 mg), cyanomethyl 3,5-dimethyl-1H pyrazole-1-carbodithioate (1) (12.68 mg, 0.03M), and N,N-dimethylformamide (1.589 mL) was prepared. The resulting mixture was degassed, sealed and heated at 100° C. for 1 hour. The volatiles were removed in vacuo to give poly(acrylic acid) at 91% conversion (based on the consumption of acrylic acid), with Mₙ 4,635, M_{w}/Mₙ 1.099.

### Example 5

### Preparation of poly(styrene) using cyanomethyl 3,5-dimethyl-1H pyrazole-1-carbodithioate (1) at 100° C.

A solution containing styrene (0.802 mL), 1,1′-azobiscyanocyclohexane (2.57 mg), cyanomethyl 3,5-dimethyl-1H pyrazole-1-carbodithioate (1) (22.19 mg, 0.0525M), and toluene (1.198 mL) was prepared. The resulting mixture was degassed, sealed and heated at 100° C. for 48 hours. The volatiles were removed in vacuo to give poly(styrene) at 52% conversion (based on the consumption of styrene), with Mₙ 6,067, M_{w}/Mₙ 1.078.

The following example serves to illustrate that with the same RAFT agent of this invention, polymers can also be made from less activated monomers.

### Example 6

### Preparation of poly(vinyl acetate) using cyanomethyl 3,5-dimethyl-1H pyrazole-1-carbodithioate (1) at 100° C.

A solution containing vinyl acetate (0.553 mL), 1,1′-azobiscyanocyclohexane (5.86 mg), cyanomethyl 3,5-dimethyl-1H pyrazole-1-carbodithioate (1) (20.28 mg, 0.048M), and ethyl acetate (1.447 mL) was prepared. The resulting mixture was degassed, sealed and heated at 100° C. for 12 hours. The volatiles were removed in vacuo to give poly(vinyl acetate) at 63% conversion (based on the consumption of vinyl acetate), with Mₙ 6,061, M_{w}/Mₙ 1.362.

The following examples serve to show that various copolymers can be made using a combination of less activated and more activated monomers, without the need to “switch” or activate the RAFT Agent. The difference between example 7 and example 8 is that in the former only 1.6% of N,N′ dimethylacrylamide relative to vinyl acetate was added whereas in the latter the ratio of N,N′dimethylacrylamide relative to vinyl acetate is 1:1. These are examples of statistical copolymers. In order to make block copolymers, one would add the monomers sequentially rather than concurrently.

### Example 7

### Preparation of poly(N,N-dimethylacrylamide)-co-poly(vinyl acetate) using cyanomethyl 3,5-dimethyl-1H pyrazole-1-carbodithioate (1) at 100° C.

A solution containing N,N′dimethylacrylamide (0.010 mL), vinyl acetate (0.553 mL), 1,1′-azobiscyanocyclohexane (5.86 mg), cyanomethyl 3,5-dimethyl-1H pyrazole-1-carbodithioate (1) (20.28 mg, 0.048M), and ethyl acetate (1.437 mL) was prepared. The resulting mixture was degassed, sealed and heated at 100° C. for 12 hours. The volatiles were removed in vacuo to give poly(N,N-dimethylacrylamide)-co-poly(acrylic acid) at 55% conversion (based on the consumption of >99% of N,N-dimethylacrylamide and 54% conversion of vinyl acetate), with Mₙ 5,353, M_{w}/Mₙ 1.230.

### Example 8

### Preparation of poly(N,N-dimethylacrylamide)-co-poly(vinyl acetate) using cyanomethyl 3,5-dimethyl-1H pyrazole-1-carbodithioate (1) at 100° C.

A solution containing N,N′dimethylacrylamide (0.309 mL), vinyl acetate (0.277 mL), 1,1′-azobiscyanocyclohexane (5.86 mg), cyanomethyl 3,5-dimethyl-1H pyrazole-1-carbodithioate (1) (20.28 mg, 0.048M), and ethyl acetate (1.414 mL) was prepared. The resulting mixture was degassed, sealed and heated at 100° C. for 12 hours. The volatiles were removed in vacuo to give poly(N,N-dimethylacrylamide)-co-poly(acrylic acid) at 81% conversion (based on the consumption of >99% of N,N-dimethylacrylamide and 62% conversion of vinyl acetate), with Mₙ 7,572, M_{w}/Mₙ 1.182.

### Example 9

### Preparation of poly(methyl methacrylate) using cyanomethyl 3,5-dimethyl-1H pyrazole-1-carbodithioate (1) at 100° C.

A solution containing methyl methacrylate (0.749 mL), 1,1′-azobiscyanocyclohexane (2.57 mg), cyanomethyl 3,5-dimethyl-1H pyrazole-1-carbodithioate (1) (11.09 mg, 0.0263M), and acetonitrile (1.251 mL) was prepared. The resulting mixture was degassed, sealed and heated at 100° C. for 4 hours. The volatiles were removed in vacuo to give poly(methyl methacrylate) at 74% conversion (based on the consumption of methyl methacrylate), with Mₙ 81,355, M_{w}/Mₙ 1.684.

### Reference Examples (Prior Art RAFT Agents)

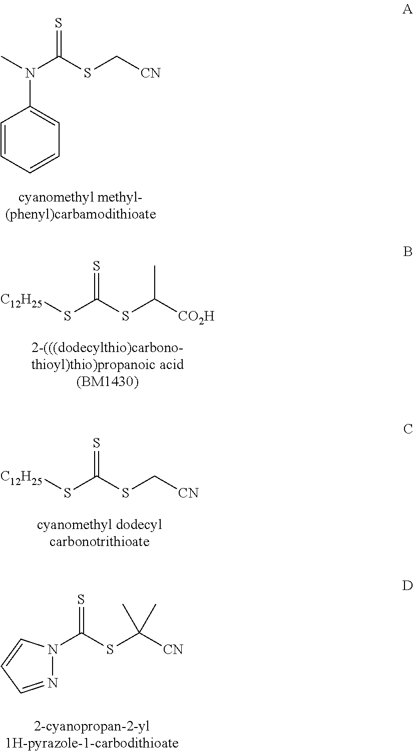

### Reference Example 1

### Preparation of poly(N,N-dimethyl acrylamide) using cyanomethyl methyl(phenyl)carbamodithioate (A) at 100° C.

A solution containing N,N-dimethylacrylamide (0.618 mL), 1,1′-azobiscyanocyclohexane (2.93 mg), cyanomethyl methyl(phenyl)carbamodithioate (A) (13.34 mg, 0.03M), and acetonitrile (1.382 mL) was prepared. The resulting mixture was degassed, sealed and heated at 100° C. for 1 hour. The volatiles were removed in vacuo to give poly(N,N-dimethyl acrylamide) at 99% conversion (based on the consumption of dimethyl acrylate), with Mₙ 14,983, M_{w}/Mₙ 1.43. This result is relatively poor compared to Example 2, in which one of the RAFT agents of this invention generated poly(N,N-dimethyl acrylamide) of similar Mn, but with a low dispersity, <1.2 reflecting properly controlled RAFT polymerisation. That is, RAFT agent (A) is a poor choice for making polymers out of more activated monomers such as N,N-dimethyl acrylamide.

### Reference Example 2

### Preparation of poly(N,N-dimethyl acrylamide) using 2-(((dodecylthio)carbonothioyl)thio)propanoic acid (B) at 100° C.

A solution containing N,N-dimethylacrylamide (0.618 mL), 1,1′-azobiscyanocyclohexane (2.93 mg), 2-(((dodecylthio)carbonothioyl)thio)propanoic acid (B) (21.04 mg, 0.03M), and acetonitrile (1.382 mL) was prepared. The resulting mixture was degassed, sealed and heated at 100° C. for 1 hour. The volatiles were removed in vacuo to give poly(N,N-dimethyl acrylamide) at 98% conversion (based on the consumption of N,N-dimethyl acrylamide), with Mₙ 10,707, M_{w}/Mₙ 1.065.

Comparing this result to Experiment 2, indicates that the RAFT agent of the current invention performs as well as a commonly used dodecyl RAFT agent, that is, results in same monomer conversion and same polymer dispersity.

### Reference Example 3

### Preparation of poly(methyl acrylate) using cyanomethyl methyl(phenyl)carbamodithioate (A) at 100° C.

A solution containing methyl acrylate (0.540 mL), 1,1′-azobiscyanocyclohexane (2.93 mg), cyanomethyl methyl(phenyl)carbamodithioate (A) (13.34 mg, 0.03M), and acetonitrile (1.460 mL) was prepared. The resulting mixture was degassed, sealed and heated at 100° C. for 1 hour. The volatiles were removed in vacuo to give poly(methyl acrylate) at 88% conversion (based on the consumption of methyl acrylate), with Mₙ 17,380, M_{w}/Mₙ 1.67. This result indicates again that RAFT agent A is a poor choice for these more activated monomers. Compare this with Example 3 in which one of the RAFT agents of this invention generated poly(methyl acrylate) of similar Mn, but with a lower dispersity, <1.2 reflecting properly controlled RAFT polymerisation. The presence of the phenyl group at the reactivity end, instead of a pyrazole group possibly played a role in that change in reactivity towards the more activated monomer.

### Reference Example 4

### Preparation of poly(methyl acrylate) using 2-(((dodecylthio) carbonothioyl)thio)propanoic acid (B) at 100° C.

A solution containing methyl acrylate (0.540 mL), 1,1′-azobiscyanocyclohexane (2.93 mg), 2-(((dodecylthio)carbonothioyl)thio)propanoic acid (B) (21.04 mg, 0.03M), and acetonitrile (1.460 mL) was prepared. The resulting mixture was degassed, sealed and heated at 100° C. for 1 hour. The volatiles were removed in vacuo to give poly(methyl acrylate) at 77% conversion (based on the consumption of methyl acrylate), with Mₙ 10,626, M_{w}/Mₙ 1.14. This result again serves to illustrate the RAFT agent of the current invention compares favourably to commonly used prior art trithiocarbonate RAFT agents such as (B), in polymerising the more activated monomer, methyl acrylates. See Example 3 again as a direct comparison.

### Reference Example 5

### Preparation of poly(N,N-dimethyl acrylamide) using cyanomethyl dodecylcarbonotrithioate (C) at 100° C.

A solution containing N,N-dimethylacrylamide (0.618 mL), 1,1′-azobiscyanocyclohexane (2.93 mg), cyanomethyl dodecylcarbonotrithioate (C) (19.05 mg, 0.03M), and acetonitrile (1.382 mL) was prepared. The resulting mixture was degassed, sealed and heated at 100° C. for 1 hour. The volatiles were removed in vacuo to give poly(N,N-dimethyl acrylamide) at 99% conversion (based on the consumption of N,N-dimethyl acrylamide), with Mₙ 12,693, M_{w}/Mₙ 1.094. Again the results of Example 1, with the use of RAFT agents of the current invention compares favourably with this reference example. That is, the pyrazole group of the current invention performs comparably to the dodecyl group of RAFT agent (C).

The below two examples serve to illustrate that the prior art RAFT agent, with similar leaving and reactive groups, may well show reactivity towards the more activated monomers, do not show any reactivity towards the less activated monomers, and in that sense are not all or multi purpose, like the RAFT agents of the current invention. The presence and positioning of the 2 methyl groups on either end of the RAFT agents appear to influence reactivities.

### Reference Example 6

### Preparation of poly(N,N-dimethyl acrylamide) using 2-cyanopropan-2-yl 1H-pyrazole-1-carbodithioate (D) at 100° C.

A solution containing N,N-dimethylacrylamide (0.618 mL), 1,1′-azobiscyanocyclohexane (2.93 mg), 2-cyanopropan-2-yl 1H-pyrazole-1-carbodithioate (D) (12.68 mg, 0.03M), and acetonitrile (1.382 mL) was prepared. The resulting mixture was degassed, sealed and heated at 100° C. for 1 hour. The volatiles were removed in vacuo to give poly(N,N-dimethyl acrylamide) at 31% conversion (based on the consumption of N,N-dimethyl acrylamide), with Mₙ 6,206, M_{w}/Mₙ 1.074.

### Reference Example 7

### Preparation of poly(vinyl acetate) using cyanomethyl 2-cyanopropan-2-yl 1H-pyrazole-1-carbodithioate (D) at 100° C.

A solution containing vinyl acetate (0.553 mL), 1,1′-azobiscyanocyclohexane (5.86 mg), 2-cyanopropan-2-yl 1H-pyrazole-1-carbodithioate (D) (20.28 mg, 0.048M), and ethyl acetate (1.447 mL) was prepared. The resulting mixture was degassed, sealed and heated at 100° C. for 12 hours. Analysis of the mixture did not show the presence of any polymer.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A RAFT agent of formula (I) 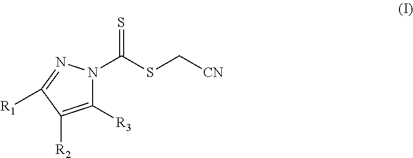
where R₁, R₂ and R₃ are each independently selected from H and optionally substituted alkyl.

2. The RAFT agent according to claim 1, wherein R₁ and R₃ are each CH₃, and R₂ is H.

3. A polymer of formula (II) or (III) 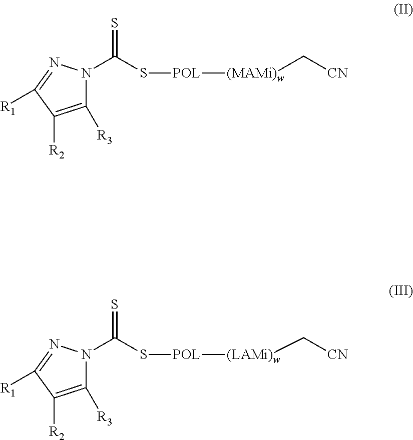
where R₁, R₂ and R₃ are each independently selected from H and optionally substituted alkyl, POL in formula (II) is a polymer chain comprising (PMAM)ₓ-(PLAM)_{y}*, POL in formula (III) is a polymer chain comprising (PLAM)_{y}-(PMAM)ₓ*, MAMi and PMAM are each independently made up of one or more RAFT reaction monomer residue units derived from one or more ethylenically unsaturated monomers of formula (IV), LAMi and PLAM are each independently made up of one or more RAFT reaction monomer residue units derived from one or more ethylenically unsaturated monomers selected from formula (V), w, x and y are each independently 0 or 1 provided at least one of x or y is 1, and * denotes the point of covalent coupling to (i) MAMi or LAMi when present, or (ii) —CH₂—CN when MAMi or LAMi are not present, 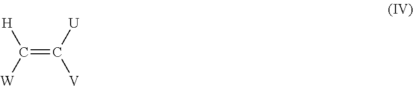
where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, 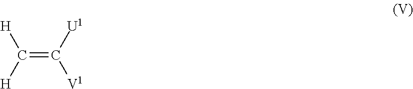
where U¹ is selected from H, C₁-C₄ alkyl or halogen; V¹ is halogen or of the form O-G where G is selected from —C(O)R¹ and —R¹, or V¹ is of the form NGG^{a} where G is as defined above and G^{a} is selected from H and R¹, G and G^{a} form together with N a heterocyclic ring, or V¹ is of the form CH₂G^{b} where G^{b} is selected from H, R¹, OH, OR¹, NR¹₂, PR¹₂, P(O)R¹₂, P(OR¹)₂, SR¹, SOR¹, and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

4. The polymer according to claim 3, wherein the one or more ethylenically unsaturated monomers of formula (IV) are selected from methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacrylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, butadiene, chloroprene, acenapthalene, vinylnapthalene, vinylbiphenyl, vinyl azlactone; 1-vinylimidazole; 2-vinylpyridine, 4-vinyl pyridine, α-methylene-γ-butyrolactone, 2-methacryloxyethyl glucoside (any anomer), and vinylferrocene.

5. The polymer according to claim 3, wherein the one or more ethylenically unsaturated monomers of formula (V) are selected from vinyl acetate, vinyl propionate; vinyl butyrate, vinyl decanoate, vinyl neodecanoate, vinyl stearate; vinyl trifluoroacetate; vinyl benzoate, vinylester-based glycomonomers, ethyl vinyl ether, vinyl chloride, vinyl fluoride, vinyl bromide, N-vinylformamide, N-vinyl-N-methylacetamide, N-vinylphthalimide, N-vinylpyrrolidone, N-vinylcaprolactam, N-vinylcarbazole, vinyl trimethylsilane, vinyltriphenylsilane, vinyltrimethoxysilane, vinyltriethoxysilane, and diallyldimethylammonium chloride.

6. The polymer according to claim 3, wherein POL in formula (II) is a block copolymer comprising a *(PMAM)-(PLAM) diblock component selected from *(polydimethylacrylamide)-(polyvinyl acetate), *(polymethyl acrylate)-(polyvinyl acetate), *(polystyrene)-(polyvinyl acetate), *(polyacrylic acid)-(polyvinyl acetate), and *(polydimethylacrylamide)-(polyN-vinyl-pyrrolidone), where * denotes the point of covalent coupling of the diblock component that is closest to a sulfur atom in formula (II).

7. The polymer according to claim 3, wherein POL in formula (III) is a block copolymer comprising a *(PLAM)-(PMAM) diblock component selected from *(polyvinyl acetate)-(polydimethylacrylamide), *(polyvinyl acetate)-(polymethyl acrylate), *(polyvinyl acetate)-(polystyrene), *(polyvinyl acetate)-(polyacrylic acid) and *(polyN-vinyl-pyrrolidone)-(polydimethylacrylamide), where * denotes the point of covalent coupling of the diblock component that is closest to a sulfur atom in formula (III).

8. The polymer according to claim 3 having a dispersity ( [symbol] ) of less than 1.4.

9. A method of preparing polymer, the method comprising polymerising under the control of a RAFT agent of formula (I) one or more ethylenically unsaturated monomers of formula (IV) and/or (V) 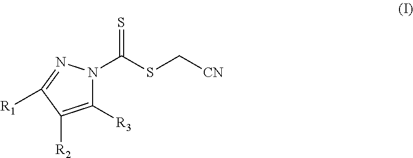
where R₁, R₂ and R₃ are each independently selected from H and optionally substituted alkyl, 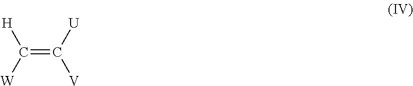
where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, 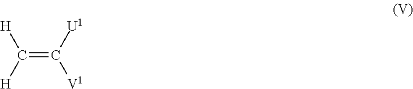
where U¹ is selected from H, C₁-C₄ alkyl or halogen; V¹ is halogen or of the form O-G where G is selected from —C(O)R¹ and —R¹, or V¹ is of the form NGG^{a} where G is as defined above and G^{a} is selected from H and R¹, G and G^{a} form together with N a heterocyclic ring, or V¹ is of the form CH₂G^{b} where G^{b} is selected from H, R¹, OH, OR¹, NR¹₂, PR¹₂, P(O)R¹₂, P(OR¹)₂, SR¹, SOR¹, and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

10. The method according to claim 9, wherein the polymer is prepared by polymerising under the control of the RAFT agent (I) a mixture of monomers of formula (IV) and (V) that comprises at least 60 mol % of monomer of formula (V), relative to monomer of formula (IV).

11. The method according to claim 9, wherein polymer is prepared by polymerising under the control of RAFT agent (I) a mixture of monomers of formula (IV) and (V) that comprises. at least 60 mol % of monomer of formula (IV), relative to monomer of formula (V).

12. A method of preparing block copolymer that comprises a polymer block derived from one or more ethylenically unsaturated monomers of formula (IV) and a polymer block derived from one or more ethylenically unsaturated monomers of formula (V), the method comprising:
(i) polymerising under the control of a RAFT agent of formula (I) one or more ethylenically unsaturated monomers of formula (IV) or (V) to form a macro-RAFT agent; and
(ii) polymerising under the control of the macro-RAFT agent formed in step (i) one or more ethylenically unsaturated monomers of formula (IV) or (V) so as to form the block copolymer,
wherein monomer polymerised in step (ii) is not of the same formula of monomer polymerised in step (i), 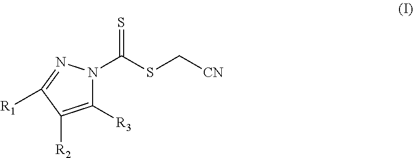
where R₁, R₂ and R₃ are each independently selected from H and optionally substituted alkyl, 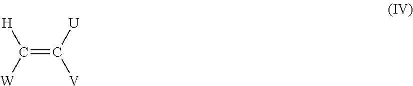
where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, 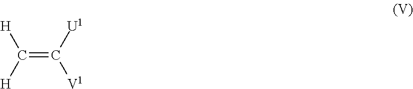
where U¹ is selected from H, C₁-C₄ alkyl or halogen; V¹ is halogen or of the form O-G where G is selected from —C(O)R¹ and —R¹, or V¹ is of the form NGG^{a} where G is as defined above and G^{a} is selected from H and R¹, G and G^{a} form together with N a heterocyclic ring, or V¹ is of the form CH₂G^{b} where G^{b} is selected from H, R¹, OH, OR¹, NR¹₂, PR¹₂, P(O)R¹₂, P(OR¹)₂, SR¹, SOR¹, and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

13. The method according to claim 9, wherein the one or more ethylenically unsaturated monomers of formula (IV) are selected from methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacrylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, butadiene, chloroprene, acenapthalene, vinylnapthalene, vinylbiphenyl, vinyl azlactone; 1-vinylimidazole; 2-vinylpyridine, 4-vinyl pyridine, α-methylene-γ-butyrolactone, 2-methacryloxyethyl glucoside (any anomer), and vinylferrocene.

14. The method according to claim 9, wherein the one or more ethylenically unsaturated monomers of formula (V) are selected from vinyl acetate, vinyl propionate; vinyl butyrate, vinyl decanoate, vinyl neodecanoate, vinyl stearate; vinyl trifluoroacetate; vinyl benzoate, vinylester-based glycomonomers, ethyl vinyl ether, vinyl chloride, vinyl fluoride, vinyl bromide, N-vinylformamide, N-vinyl-N-methylacetamide, N-vinylphthalimide, N-vinylpyrrolidone, N-vinylcaprolactam, N-vinylcarbazole, vinyl trimethylsilane, vinyltriphenylsilane, vinyltrimethoxysilane, vinyltriethoxysilane, and diallyldimethylammonium chloride.

15. The method according to claim 9, wherein the so formed polymer or copolymer has a dispersity ( [symbol] ) of less than 1.4.
